(19) United States
(12) Patent Application Publication
(10) Pub. No.: US 2015/0159201 A1
(43) Pub. Date: Jun. 11, 2015

(54) METAGENOMIC ANALYSIS OF SAMPLES

(71) Applicant: RTG NZ Holdings Limited, Hamilton Central (NZ)

(72) Inventors: John Gerald CLEARY, Hamilton (NZ); Sean Allistair IRVINE, Hamilton (NZ); Kurt GAASTRA, Hamilton (NZ)

(73) Assignee: RTG NZ Holdings Limited

(21) Appl. No.: 14/564,810

(22) Filed: Dec. 9, 2014

Related U.S. Application Data
(60) Provisional application No. 61/914,368, filed on Dec. 10, 2013.

(51) Int. Cl. C12Q 1/68; G06F 19/22

(52) U.S. Cl. CPC C12Q 1/689; G06F 19/22; C12Q 2600/16; C12Q 2600/158

(57) ABSTRACT

Methods and systems for evaluating genomic sequences are described. The methods include approaches for evaluating the prevalence of genomes in a source including changes over time based on the prevalence of segments in samples obtained from the source, and may additionally rely on the prevalence of segments in reference genomes and an estimated genome population distribution of the sample.

This application claims the benefit of U.S. Provisional Patent Application No. 61/914,368, filed Dec. 10, 2013, the entire contents of which are incorporated herein by reference for all purposes. The entire contents of U.S. patent application Ser. No. 13/925,704, filed Jun. 24, 2013, entitled “POPULATION BASED METHOD OF EVALUATING GENOMIC SEQUENCES,” are incorporated herein by reference for all purposes.

The present invention relates to methods and systems for the metagenomic analysis of samples. More particularly, although not exclusively, the invention relates to methods for evaluating the correlation between a sample containing multiple genomes and reference genomic sequences where the proportions of different genomes in the sample are unknown.

In nature there are numerous patterns that can be interpreted as sequences of discrete units. In biology, the sequence of nucleotides in DNA or RNA, and the sequences of amino acids in proteins are of particular interest. In DNA, sequences consist of discrete units which may take on one of the values A, C, G, T, while in RNA sequences, the values are A, C, G, and U. Proteins represent a more complicated sequence, as individual units may be one of 21 or more amino acids—in general 22 amino acids.

Sequencing machines are used to produce a machine readable encoding of such biological sequences. These machines use a variety of techniques to interpret the molecular information, and may introduce errors into the data in both systematic and random ways. Errors can usually be categorised into substitution errors, where the real code is substituted with an incorrect code (for example A swapping with G in DNA), or so called indel errors (insertion/deletion), where a random unit is inserted (for example AGT becoming AGCT in DNA) or deleted (for example AGTA becoming ATA).

A sequencing machine may produce a number of ‘reads’, where each read is a small segment of a genome sequence sample, for example a 3 billion unit long DNA collection of chromosomes may be sequenced into reads of only 100 units in length. Due to the method of generating the reads, the original position of each read against the original sequence is unknown, and so mapping and alignment techniques can be used to determine the original location of the reads. Typically alignment will need to take into account that the direction of the reads is also unknown.

Often a sample will contain a mixture of DNA from different organisms (e.g. a sample from a human may contain human DNA, bacterial DNA, viruses etc.). Environmental samples may typically include hundreds or thousands of different genomes, or in some cases tens or hundreds of thousands or even millions.

WO2009/085473 discloses probabilistic matching techniques but does not utilise any population distribution information. Reads are treated as correct and no substitution or indel errors are considered. The probabilistic matching is based simply on the probability of reads matching a reference genome with no appreciation of genome population distribution.

Traditional analysis techniques seek to identify the presence or absence of a specific genomic sequence in isolation. This loses information value that may be obtained from the other nucleotide sequences in the sample. It can be difficult to evaluate the significance of read/reference genome correlations when the relative proportions of reference genomes is unknown. Additionally, there is a need for monitoring of changes in a population over time. Although sequence data can be obtained from samples, its application in time-resolved monitoring applications has been limited, including in medical contexts such as the early stages of development of an infection.

It would be useful to evaluate to improve the accuracy of correlation between samples and reference sequences and/or of the determination of changes of prevalence of sequences in a time series of samples from a source using population information or to at least provide the public with a useful choice.

According to a first aspect there is provided a method of evaluating a change in prevalence of a plurality of genomes in a source over time, comprising using a processing engine to optimise estimated proportion values for the plurality of genomes in at least a first sample obtained from the source at a first time and a second sample obtained from the source at a second time, based on input data comprising the prevalence of segments in at least the first and second samples and the prevalence of the segments in the plurality of genomes, thereby producing optimised proportion values for the prevalence of the plurality of the genomes in at least the first and second samples.

Additional aspects are set forth elsewhere in this description and in the appended set of claims.

As used herein, “segments” may comprise, e.g., reads obtained from a nucleic acid sequencing apparatus. In some embodiments, some or all of the reads are paired end reads; in other embodiments, the reads are not paired end reads. Alternatively, segments may comprise fragments of a given length (“K-mers”), Using K-mers that are shorter than reads can allow matching to genomes of related species. For example, in some embodiments, K-mers with a length less than or equal to 64, 50, 45, 40, 35, 30, 35, 20, or 15 bases are used. In some embodiments, the length of the K-mers also have a length greater than or equal to 10, 15, 20, 25, 30, 35, 40, or 50 bases. This approach may be useful when many of the genomes represented in a sample are not from the same species as the reference genomes.

A probability function may be employed to optimise the proportion values using an iterative process. The probability function may be a Poisson distribution. The probability function may be optimised by moving the iteration in a direction in which the derivative of the probability function is most strongly increasing. The iteration step size may be determined based on the second derivative of the probability function. The iteration step size is determined based on the second derivative of the probability function in the direction that the derivative is most strongly increasing.

The evaluation may be performed for variants of the segments also which may include indels and/or substitutions. The evaluation may also be performed for the reverse complement direction of the segments or variants also. Each variant may have a weighting representing the likelihood of the respective variant occurring. The likelihood may be based at least in part on the apparatus employed or the chemistry of the sample. For example, segments rich or poor in C and/or G nucleotides are selectively favored or suppressed in some sequencing machines. For example, runs of the same consecutive nucleotide can be over or under counted in some sequencing machines. The likelihood of a variant can be adjusted using previously observed rates of these events for a particular sequencing machine and chemistry.

A table of exemplary transition probabilities is shown in Table 1 below. The exemplary transition probabilities represent prior probabilities of X to Y transitions.

TABLE 1

| X to Y | Probability |
|---|---|
| A to A | 0.99897133 |
| T to T | 0.99897329 |
| C to C | 0.99841451 |
| G to G | 0.99841501 |
| A to G | 0.00030100 |
| T to C | 0.00029994 |
| C to T | 0.00037643 |
| G to A | 0.00037624 |
| A to C | 0.00007084 |
| T to G | 0.00007121 |
| C to A | 0.00009541 |
| G to T | 0.00009555 |
| A to T | 0.00005627 |
| T to A | 0.00005571 |
| C to G | 0.00010390 |
| G to C | 0.00010356 |

Segments that do not correlate with the plurality of reference genomes (“unmapped segments”) may be categorised based on paired end reads or not paired end reads etc. Categorisation information may be used to refine proportion values. For example, when a segment that does not correlate with the plurality of reference genomes has a corresponding paired end segment that does correlate, the confidence that the correlated segment was correctly assigned or mapped may be reduced. A similar reduction in confidence may be applied when the members of a pair of paired end reads correlate with different reference genomes. Unmapped segments also provide information about the proportion of the sample which is not represented in the reference genomes. Thus, their abundance can be used to reduce the proportion of known genomes when analysing proportion values with respect to the overall sample.

Unmapped segments may be assembled into composite segments consisting of two or more segments of the same category and the composite segments may be utilised to refine proportion values.

A plurality of algorithms may be employed to determine proportion values. Examples of algorithms which can be used are discussed later in this specification. The algorithms are applied in an order depending upon processing metrics including one or more of: the speed of each algorithm; the precision of each algorithm; the number of segments in the sample; the number of segments in the genomes; the number of genomes; the nature of the sample; the available processing resource; and the required confidence level for the proportion values. Historical probability information of prior samples may also be utilised to assess correlation. For example, historical observations can be used as a starting point or initial guess for proportion values. This approach may be applicable, e.g., for a time series of samples from a site or group of related sites where proportion values from earlier samples have been determined.

In some embodiments, the methods comprise comparing the relative abundance as a function of time of one, two, three or more components in samples from a source. The components may be genomes or organismal species (e.g., microbes, such as bacteria, including pathogenic bacteria). The source may be a host organism, such as a mammal, e.g., a human, including human infants. Thus, it is possible to determine if the amount(s) of one or more organisms within or on the source (which amount(s) can be determined using the proportion value(s) of its (their) genome(s)) has increased significantly in a later (e.g., second) sample relative to an initial or earlier (e.g., first) sample. In some embodiments, methods comprise determining whether a component in the sample has changed (e.g., increased) by a factor of greater than or equal to 1.2, 1.5, 2, 3, or more during a time period between the taking of any two samples; decreases of equivalent magnitude may also be detected. Such determination may be useful in medical applications, e.g., detecting an incipient or developing infection in a host organism. In some cases, the increase in abundance of a pathogen can be detected from host organism samples prior to the reporting and/or observation of disease symptoms. Such detection can provide extra time to treat or mitigate the infection before adverse consequences such as severe or life-threatening illness develop. In some embodiments, the component that has changed in abundance comprises at least one pathogenic bacterial genome. In some embodiments, the component that has changed in abundance comprises a Streptococcus genome, a Serratia genome, a Clostridium genome, a Staphylococcus genome, or an Escherichia coli genome. In some embodiments, the component that has changed in abundance comprises a Group B Streptococcus genome, a Streptococcus agalactiae genome, a Serratia marcescens genome, a Clostridium difficile genome, a coagulase-positive Staphylococcus genome, a Staphylococcus aureus genome, and an Escherichia coli pathogenic strain genome. It is understood from the discussion herein that embodiments according to this disclosure can provide solutions to technological problems. The technological problems include, e.g., rapid detection of a change in the amount of one or more organisms, such as a microbe, in or on a source, such as a host. The change may be qualitative (e.g., from not present to present) or quantitative (e.g., a two-fold increase). As discussed in additional detail above and below, the organism can be, e.g., a pathogenic bacterium, and the host can be, e.g., a human infant, such as a newborn of an age less than or equal to 3, 2, or 1 months, 4, 3, 2, or 1 weeks, or 6, 5, 4, 3, 2, or 1 days. The technological problems that can be solved by certain embodiments also include, e.g., providing extra time to treat or mitigate an infection before adverse consequences such as severe or life-threatening illness develop in the host. These solutions may represent improvements over existing alternatives in terms of speed, specificity, and/or sensitivity. Sensitivity can refer to the number of different organisms that can be detected, and/or to the threshold for detection of an organism. The problems that can be solved by certain embodiments solved also include the environmental detection of one or more organisms, e.g., in a neonatal intensive care unit, or other health care facility, leading to quicker response and action against detected organisms.

In some embodiments, the methods comprise obtaining sequence data from a sample from a subject, who can be a human subject. The subject may be asymptomatic, or be a subject for whom symptoms have not been reported. The subject may be, for example, under the care of a physician, in need of health screening, or in need of surveillance for infection. For example, the subject may have a compromised or repressed immune system (e.g., due to immunosuppressive drug therapy, exposure to radiation, or an illness such as AIDS) or may have an immune system at a limited stage of development, e.g., an infant, such as an infant of less than or equal to five, four, three, two, or one week of age. In some embodiments, the infant is premature, e.g., born at a gestational age of less than or equal to 37, 36, 35, 34, 33, 32, 31, 30, 29, 28, 27, 26, 25, 24, or 23 weeks. In some embodiments, the infant has been admitted to a neonatal intensive care unit or is being treated in a neonatal intensive care unit. In some embodiments, the infant is receiving respiratory assistance and/or has respiratory difficulty. The respiratory difficulty may exist in the absence of reported or observable symptoms of any infectious disease. Alternatively, whether or not the infant is receiving respiratory assistance and/or has respiratory difficulty, the infant may have exhibited a change in heart rate variability, such as bradycardia or a level of consistency in the heart rate indicative of incipient or oncoming sepsis. Heart rate variability, the correlation of low variability with conditions such as sepsis, including in infants, and the monitoring thereof is discussed, e.g., in U.S. Pat. No. 6,804,551, which is incorporated herein by reference. Such change in heart rate variability can be observed, e.g., during the time in which samples are being obtained or after samples have been obtained. In some embodiments, the infant has a birth weight of less than or equal to 2 kg, 1.9 kg, 1.8 kg, 1.7 kg, 1.6 kg, 1.5 kg, 1.4 kg, 1.3 kg, 1.2 kg, 1.1 kg, or 1 kg.

Samples may be obtained at various frequencies. For example, the methods may comprise taking at least 1, 2, 3, 4, 5, 6, 7, 8, 9, 10, 11, 12, 18, 24, or more samples per day, for a period of 1, 2, 3, 4, 5, 6, 7, 8, 9, 10, 11, 12, 13, 14, 15, 20, 21, 25, 28, 30, or more days. Alternatively, the methods may comprise taking at least 1, 2, 3, 4, 5, 6, 7, 8, 9, 10, 11, 12, 13, 14, 21, 28, or more samples per week, for a period of 1, 2, 3, 4, 5, 6, 7, 8, 9, 10, 11, 12, 13, 14, 15, 18, 20, 24, 30, or more months.

The type of sample to be obtained from a subject can be chosen from, e.g., fecal, urine, saliva, buccal swab, or skin samples. In some embodiments, the method involves obtaining two or more different types of sample from the same source at the same or different times; thus, for example, a urine sample and a fecal sample can be obtained. The population of organisms (e.g., bacteria) resident on the skin of a subject can be assessed, e.g., by swabbing or washing with a suitable fluid to separate organisms from the skin without causing a wound. One skilled in the art is familiar with techniques for obtaining sequence data from such samples. In additional embodiments, a sample is obtained from a second source, which may be genetically related to the first source (e.g., a parent, sibling, or child). Alternatively or in addition to being genetically related, the second source may have been exposed to similar environmental conditions (e.g., being in the same hospital department or hospital room). As discussed in this disclosure, proportion values for genomes can be determined for one, two, three, or more samples from the second source (it being possible for the samples to be taken at different times, as discussed in the preceding paragraph). It is then possible to compare results, such as the change in one or more proportion values, from the first source to the proportion values and/or the change in one or more proportion values from the second source. Where the two sources have differing observations (e.g., disease symptoms), the comparison of proportion values may indicate which genome or genomes are likely responsible. By way of a simple example, if the first source shows a disease symptom and shows an increase in both genome A and genome B, while the second source does not show a disease symptom and shows an increase in genome A but not genome B, then it can be inferred that the organism associated with genome B is the likely cause of the symptom.

In some embodiments, the plurality of genomes comprises one, two, three, or more of a Streptococcus genome, a Serratia genome, a Clostridium genome, a Staphylococcus genome, and an Escherichia coli genome.

In some embodiments, the plurality of genomes comprises one, two, three, or more of a Group B Streptococcus genome, a Streptococcus agalactiae genome, a Serratia marcescens genome, a Clostridium difficile genome, a coagulase-positive Staphylococcus genome, a Staphylococcus aureus genome, and an Escherichia coli pathogenic strain genome.

In some embodiments, the methods further comprise treatment and/or quarantine of the source. For example, at least one medicament (e.g., antibiotic, antifungal, or antiparasitic) or an effective regimen of one or more medicaments (e.g., antibiotics, antifungals, or antiparasitics) can be administered to the source. The selection of appropriate antibiotic regimens to treat various pathogens is known to those skilled in the art. Alternatively or in addition, the source may be placed under quarantine and/or isolated from other individuals who may be susceptible to infection. These interventions may be predicated on a finding that a certain genome is prevalent or has increased in prevalence over time as shown by one or more samples from the source.

Sample segments may be translated into another form and compared to reference segments in that form. For example DNA segments may be converted to protein segments and compared to a reference library of protein segments. In this way, proportion values for individual proteins or pathways (such as metabolic or signalling pathways) involving a plurality of proteins can be determined, instead of or in addition to determining proportion values of genomes.

The correlation between sample genomes and reference genomes obtained may be displayed by displaying regions of correlation between reads and reference sequences. A user may be able to modify correlation parameters such as read length; population distribution; one or more thresholds; read variants to vary the displayed correlation. Correlation may be displayed by colour or some other optical attribute.

The abundance of one or more genome in biological samples (e.g. genome equivalents per unit mass or volume, or mass of genomic material attributable to the one or more genome per unit mass or volume) may be determined based on the proportion values and the concentration of genomic material in the samples. The samples may be data samples, i.e., samples comprising biological sequence segments stored in a computer-readable medium.

A user may modify one or more correlation parameters to investigate relationships. A user may set a global probability range and explore values within that range. Alternatively a user may modify read length; population distribution; one or more thresholds or read variants. The degree of correlation may be indicated by a visual attribute of each region, such as colour.

There is also provided a first system for determining the change in genomic population distribution of a source over time including:
a. a sequencer to obtain reads from at least a first sample obtained from the source at a first time and at least a second sample obtained from the source at a second time and;
b. a first database of reference genome segments; and
c. a first processing engine for determining proportion values of the reference genomes occurring in the sample by optimising the proportion values.

The processing engine may apply a number of algorithms in an order depending upon processing metrics such as: the number of segments in the samples; the number of segments in the genomes; the number of genomes; the nature of the samples; the available processing resource; the required confidence level for the proportion values.

According to a further aspect there is provided a distributed processing system including a first system as above and a second system in communication with the first system, the second system including:
a. a second database storing reference genome segments not stored in the first database; and
b. a second processing engine for processing segments received from the first system using the second database to develop proportion values.

Processing may be transferred from the first system to the second system when the required processing exceeds a metric of the first processing system such as processing capacity; estimated job run time; required proportion values confidence level etc.

The second processing engine may utilise algorithms and/or genome segments not available to the first processing engine.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1 shows a sequencing-processing system for determining the genomic population distribution of at least a first sample obtained from a source at a first time and a second sample obtained from the source at a second time, the system comprising a sequencer 1 to obtain read from at least first and second samples, the samples comprising nucleic acid, which is operably linked to a processing system 2 that can comprise at least one user interface element chosen from a display 7, a keyboard 8, and a mouse 9, and at least one computer 3 comprising a storage 4 comprising a first database comprising reference genome segments, a bus system 5, and a processing engine 6 configured to determine optimised proportion values of the reference genomes occurring in at least the first and second samples by optimising estimated proportion values and to determine whether the optimised proportion value of at least one reference genome for the second sample is changed relative to the optimised proportion value of the reference genome for the first sample.

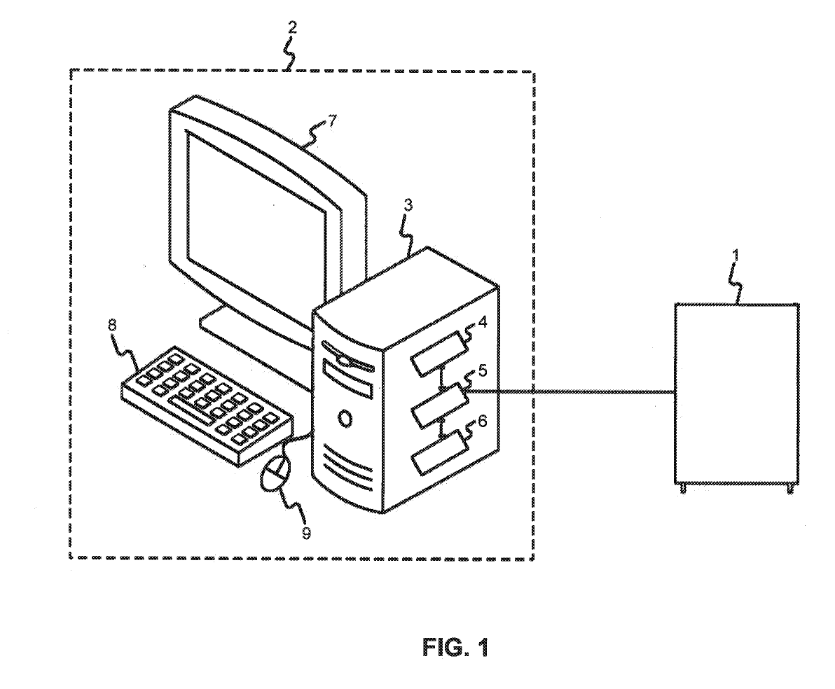

FIG. 2 shows a distributed processing system comprising the sequencing-processing system of FIG. 1 in communication (e.g., via network 10) with a second system 12, wherein the second system can comprise at least one user interface element chosen from a display 17, a keyboard 18, and a mouse 19, and at least one computer 13 comprising a storage 14 comprising a second database comprising reference genome segments not stored in the first database, a bus system 15, and a second processing engine 16 for processing segments received from the first system using the second database to develop proportion values.

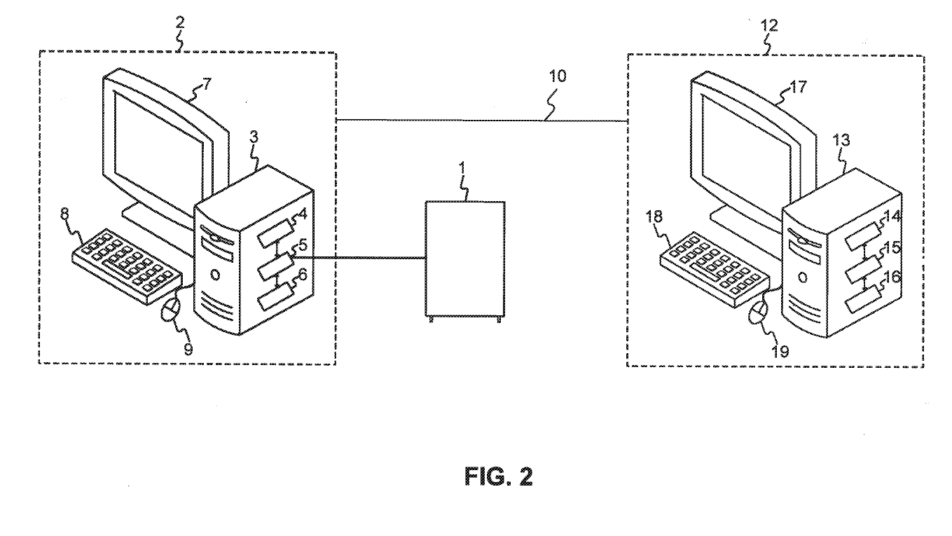

### DETAILED DESCRIPTION

To facilitate the understanding of this invention, a number of terms are defined below. Terms not defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

In this specification the term “segment” is used to refer to part of a biological sequence (e.g. a genomic, mRNA, or protein sequence) that may be a “read” from a sequencer or a part of a known genomic, mRNA, or protein sequence or some other part of a larger sequence.

A sequencer includes a system for individual molecule sequencing, which includes but is not limited to the 454 FLX™ or 454 TITANIUM™ (Roche), the SOLEXA™/Illumina Genome Analyzer (IIlumina), the HELISCOPE™ Single Molecule Sequencer (Helicos Biosciences), and the SOLID™ DNA Sequencer (Life Technologies/Applied Biosystems) instruments), as well as other platforms such as those of Intelligent Biosystems, Oxford Nanopore Technologies, and Pacific Biosciences. Sequencers also include bulk (e.g., Sanger) sequencing systems, such as the ABI 96-capillary 3730XL Sequencer. The structure and components of a sequencer vary depending on the approach employed by the sequencer. Generally, however, sequencers include a chamber for holding a sample, components for reacting and/or resolving nucleic acid molecules in the sample, one or more sensors for detecting product nucleic acid molecules and/or resolved nucleic acid molecules, and an interface for providing sequencing data to a processing engine such as a computer.

The term “processing engine” as used herein refers to a device or system, such as a computer, which can perform calculations, data manipulation, and the like; neither the human mind nor a human with writing instrument and paper are a processing engine for purposes of this specification.

A generalised method of using genomic population distribution information of a sample will be described to illustrate key principles. In this generalised example a physical sample containing multiple genomes is sequenced to produce a series of reads (segments). Based on the number of occurrences of each unique read there will be a prevalence factor associated with each read. For a library of known genomes each genome will have associated prevalence factors for each unique read (segment). By adjusting estimated proportions of each genome within the sample a “best fit” can be calculated. “Best fit” generally refers to the optimal set of proportion values among the sets evaluated by the method, not necessarily the optimum among all possible sets of proportion values.

As illustrated in a simplified example in Table 2 below a first estimate of the population distribution of the genomes listed under the “Species” heading is estimated in iteration ITER1. For this population distribution a probability function can calculate a global probability L based on the prevalence of segments in the sample (“reads”) and the known prevalence of those segments in the respective genomes.

TABLE 2

| Species | ITER1 | ITER2 | ITER3 | ITER4 | ITER5 |
|---|---|---|---|---|---|
| A | 0.8 | 0.85 | 0.84 | 0.84 | 0.84 |
| B | 0.1 | 0.05 | 0.06 | 0.06 | 0.065 |
| C | 0.01 | 0.01 | 0.01 | 0.01 | 0.01 |
| D | 0 | 0 | 0 | 0.01 | 0.01 |
| E | 0.09 | 0.09 | 0.09 | 0.08 | 0.085 |
| L | 0.9 | 0.91 | 0.92 | 0.93 | 0.935 |

In a second iteration ITER2 the population distribution is adjusted and a new global probability L is calculated. The population distribution is adjusted through successive iterations to ITER5 where an optimised global probability value is achieved. A global probability value can be considered optimised based on achieving a predetermined threshold confidence level, or based on convergence of iterations within a specified level of tolerance.

The first estimate or initial guess may be obtained in any suitable manner, for example, using a uniform distribution, using a historically observed set of proportion values or an average or median of a set of such sets, using proportion values based on frequencies of reads that map uniquely to a reference genome, or using proportion values from overall frequencies of reads mapping to each reference genome. Thus, methods to form the first estimate of the population distribution include the weighted proportion of the segments in the genomes; the proportion of segments uniquely assigned to the genomes; or a uniform distribution. When using the weighted proportion of the segments in the genomes, if a segment r hits s genomes, then it contributes r/s to each of the genomes it hits and 0 to all the others. The initial estimates are then the sum of all these r/s across all r, divided by the total number of segments. In some embodiments, the number of hits per genome may be assumed to be non-zero (e.g., 1 hit), which may improve convergence of iterative methods.

This is a highly simplified example to illustrate the general principle. It will be appreciated that by utilising additional information and potential constraints relating to non-target genomic sequences a population distribution may be obtained that enables a more accurate determination of the prevalence of a target genome to be determined as information as to non-target genomes may constrain the range of probabilities for target genomes. This may be particularly useful where, in addition to proportion values, the likelihood that one or more particular genomes are present in a sample and/or a confidence interval for one or more of the proportion values is to be determined.

Whilst population distribution could be estimated by a brute force technique this would be very computationally inefficient where high numbers of genomes are present. The number of known genomes is increasing quickly, so a desirable attribute of a method would be one that scales up to handle potentially hundreds of millions of species. A brute force method may calculate all values of a probability function for stepped proportions of every genome and refine searching at smaller steps near maxima of each iteration. Where very large numbers of genomes are involved (often tens of thousands in e.g. physical samples found in nature and data samples derived from such physical samples) the computational requirements for a brute force approach may be prohibitive.

A preferred approach evaluates the correlation between a plurality of genomes and segments of a sample based on the prevalence of the segments in the sample, the prevalence of the segments in the genomes and an estimated proportion value for each genome in the sample by optimising the proportion values using a probability function.

A “hill climbing” technique is one way to optimise the probability function to obtain optimised proportion values. The problem for estimating genome frequencies can be formulated in a way that leads to a function f(x1, x2, x3, . . . xn) where n is the number of genome frequencies to be estimated and x1 xn are the frequencies. We want to find the values of x1, x2, x3, . . . xn such that f is as large as possible (its maximum). Where f has a single maximum (peak) in its value (i.e. no small local maxima (peaks) which are lower than the main peak) the following approach may be employed. The derivative of the function represents slope and the second derivative represents the curvature of f with respect to each of x1, x2, x3, . . . xn. As n can be large (in the thousands and potentially in the millions) there can be practical difficulties in finding the peak and ensuring numerical stability unless a directed approach is employed. The peak is the place where all the derivatives are zero (the top of a hill is flat, i.e. not sloped upward or downward) although the second derivatives will usually not be (the peak has a curve in the ground, i.e. curvature is present).

To find a maximum of function f computation starts at a start position (some value of x1, x2, x3, . . . xn) and the derivative at this point is calculated and then successive iterations move in the direction where the derivative is most strongly increasing (i.e. move directly upslope). To be clear, the derivative calculated in this step is a vector quantity, as opposed to a scalar derivative. This can be repeated through several iterations. If the derivative is below a selected threshold then an acceptable solution for the proportion values x1 to xn has been found. The threshold may be evaluated with respect to a scalar determined from the partial derivatives, e.g., the sum of the squares of the partial derivatives or the square root thereof; or the threshold may be evaluated with respect to each partial derivative individually. The step size can be computed using both the derivative and the second derivative using Newton's method. This method both calculates the direction to move and how far you move. However, there may be difficulties with numerical stability in some cases, especially when the first partial derivatives are almost zero (i.e. close to the top of a hill where the slope gets relatively flat) and when the first partial derivatives in different directions are very different (i.e. at a ridge where the top of the ridge slopes gently and the slopes to either side are very steep).

A further refinement uses the derivative to select a direction to move. The direction to move may be determined using the first derivative of f as described above. Then a new function g(xd) representing a slice through f in the chosen direction may be used to determine step size. In this way there is a single number xd which may be varied to find a maximum (this isn't necessarily the top of the hill—just the best we can do in the direction chosen). The first and second derivatives of g may then be calculated. At the chosen starting point, which is set so that xd=0, we know that the first derivative of g(xd) is positive. We then choose a small step and evaluate g(xd) at that point and keep doubling the step until g(xd) becomes negative (i.e. where we have passed the point where it is 0). From these values can be determined lower and higher values for xd and it is known that the best value for g (the point where the derivative is zero) lies between these two values. Newton's method (a simple version that only varies one number) may then be used to find the peak for g(xd). Once this value is obtained a new position x1, x2, x3, . . . xn is obtained for the next iteration. This continues until the first derivative of function f is below a selected threshold value.

By way of simple analogy in three dimensional space the form of the surface can have a significant impact upon the reliability of the optimisation. For example if the surface has a large plateau then there are a number of proportions close to the optimised value with very similar probabilities. Using a graphical interface the nature of the topology may be presented to a user. This may require dimension reduction techniques to create a visual display that is meaningful to a user. It could be an arbitrary representation in which the values lie within a desired global probability range. However, continuing with the simplified 3D example a user may view a hill with a large plateau. The user may be able to navigate around the plateau using a mouse or other input device. As a user navigates to different points certain information associated with the selected point may be displayed—for example the proportions of two target genomes. This enables a user to explore a range of proportions with similar probabilities to see if there is a surprising result or a result that a user can determine to be a better result due to user knowledge (i.e. knowledge that certain proportions are impossible or unlikely or are likely).

A more detailed discussion of the mathematical basis for probabilistic optimisation is provided below. In addition to the hill climbing technique described above the use of a Hessian matrix is also discussed.

### Hill Climbing Example

h is an index over units that can potentially be matched against the genomes. These might be individual exactly matching hashed words or best matching reads.

Definition 1. nₕ
nₕ=number of occurrences of h in the reads,

Care is needed as we must deal correctly with the case when nₕ=0; that is, a unit that is in one or more genomes but which is not seen in the sample.

Definition 2. m_{h,i}
m_{h,i}=number of occurrences of h in the ith genome.

Definition 3. \hat{r}i
\hat{r}i=the estimate of the number of occurrences of the ith genome in the reads.

That is Pi is an unnomialized count of the number of individuals with the genome in the sample.

Definition 4. ph

$${ρ}_{h} = {\sum }_{k} {m}_{h , k} {r}_{k} .$$

Definition 5. P

$$P = {\prod }_{h} \frac{{e}^{- {ρ}_{h}} {ρ}_{h}^{{n}_{h}}}{{n}_{h} !} .$$

The global probability of a particular assignment of estimates assumes a Poisson distribution.

Definition 6. L

$$L = - ln ( P ) = - {\sum }_{h} - {ρ}_{h} + {n}_{h} ln ( {ρ}_{h} ) - ln ( {n}_{h} ! ) .$$

L is the negative log of the global probability of a particular assignment of estimates. We will be looking for the minimum which can be found by setting the partial derivatives to 0. Using the negative means that in later steps the Hessian will be a positive definite number (rather than negative definite) which fits the usual conventions in the literature.

We will alter the convention for the summations here. Let h range over units where nₕ>0 and h over all units, that is nₕ′≧0. Also note that the terms ln(nₕ!) are constant (independent of \hat{r}i) so can be ignored for the purposes of minimization and that nĥ′ ln(ρₕ′)=0 when nₕ′=0.

$$\begin{matrix}L = {\sum }_{{h}^{′}} {ρ}_{{h}^{′}} - {\sum }_{h} {n}_{h} ln ( {ρ}_{h} ) \\ = {\sum }_{{h}^{′}} {\sum }_{k} {m}_{{h}^{′} , k} {r}_{k} - {\sum }_{h} {n}_{h} ln ( {ρ}_{h} ) \\ = {\sum }_{k} {r}_{k} {\sum }_{{h}^{′}} {m}_{{h}^{′} , k} - {\sum }_{h} {n}_{h} ln ( {ρ}_{h} ) \\ = {\sum }_{k} {r}_{k} {l}_{k} - {\sum }_{h} {n}_{h} ln ( {ρ}_{h} )\end{matrix}$$

where lₖ=Σ_{h′}m_{h′,k} and is in most cases well approximated by the length of the genome k.

Also note that if we sum over all occurrences of h then the value of L is unchanged and effectively each nₕ=1. So our final expression for L the function to be minimized is:

$$L = {\sum }_{k} {r}_{k} {l}_{k} - {\sum }_{h} ln ( {ρ}_{h} )$$

There are many techniques for minimizing functions with varying complexity, robustness and execution time. Many of them require knowledge of the first and/or second derivatives of the function. The first point to note is that the minimum can be defined by the point(s) where the first partial derivatives are zero.

$$\begin{matrix}\frac{\partial L}{\partial {\hat{r}}_{i}} = {\sum }_{k} \frac{\partial }{\partial {\hat{r}}_{i}} {r}_{k} {l}_{k} - {\sum }_{h} \frac{\partial }{\partial {\hat{r}}_{i}} ln ( {\sum }_{k} {m}_{h , k} {r}_{k} ) \\ = {l}_{i} - {\sum }_{h} \frac{{m}_{h , i}}{{ρ}_{h}} \\ = 0\end{matrix}$$

Minimizing Technique 1: Direct Non-Linear

A direct non-linear technique may proceed in two steps: first a direction in which to move is determined and then the minimum for L is found along that direction. This procedure is repeated until some acceptable convergence criterion has been satisfied. In terms of the literature for solving such equations the first derivatives are called the Jacobian and written

$${J}_{i} = \frac{\partial L}{\partial {\hat{s}}_{i}} .$$

The basic principle is that the direction should be opposite to J, that is Δ=−J. Then the problem is to find the minimum at δΔ where δ≧0.

Minimizing Technique 2: Multi-Dimensional Newtons

Solving this requires the additional constraints that \hat{r}ᵢ≧0 which would need the use of Lagrange multipliers. To simplify this and remove the need to explicitly deal with these constraints, the problem can be reformulated in terms of the variables ŝᵢ.

Definition 7. ŝᵢ

ŝᵢ=ln(\hat{r}ᵢ).

Recalling that ρₕ=Σₖm_{h,k}e^{ŝ}^{ₖ} we can reformulate the problem to

$$\begin{matrix}\frac{\partial L}{\partial {s}_{i}} = {\sum }_{k} \frac{\partial }{\partial {\hat{s}}_{i}} {e}^{{\hat{s}}_{k}} {l}_{k} - {\sum }_{h} \frac{\partial }{\partial {\hat{s}}_{i}} ln ( {ρ}_{h} ) \\ = {e}^{{\hat{s}}_{i}} {l}_{i} - {\sum }_{h} \frac{\partial }{\partial {\hat{s}}_{i}} ln ( {\sum }_{k} {m}_{h , k} {e}^{{\hat{s}}_{k}} ) \\ = {\hat{r}}_{i} {l}_{i} - {\sum }_{h} \frac{{m}_{h , i} {e}^{{\hat{s}}_{i} }}{{ρ}_{h}} \\ = {\hat{r}}_{i} {l}_{i} - {\sum }_{h} \frac{{m}_{h , i} {\hat{r}}_{i}}{{ρ}_{h }} \\ = {\hat{r}}_{i} ( {l}_{i} - {\sum }_{h} \frac{{m}_{h , i}}{{ρ}_{h}} ) \\ = 0\end{matrix} \qquad$$

In terms of the literature for solving such equations the first derivatives are called the Jacobian and written

$${J}_{i} = \frac{\partial L}{\partial {\hat{s}}_{i}} .$$

Some minimization techniques also require the second derivatives which forms a matrix called the Hessian matrix which is written:

$$\begin{matrix}{H}_{i , j} = \frac{{\partial }^{2} L}{\partial {\hat{s}}_{i} \partial {\hat{s}}_{j}} \\ = \frac{\partial }{\partial {s}_{j}} {e}^{{\hat{s}}_{i}} {l}_{i} - {\sum }_{h} {m}_{h , i} \frac{\partial }{\partial {\hat{s}}_{j}} \frac{{e}^{{\hat{s}}_{i}}}{{ρ}_{h}}\end{matrix}$$

Split into two cases when i=j and i≠j,
i≠j

$$\begin{matrix}{H}_{i , j} = {\sum }_{h} {m}_{h , i} \frac{{e}^{{\hat{s}}_{i}}}{{( {\sum }_{k} {m}_{h , k} {e}^{{\hat{s}}_{k}} )}^{2}} {m}_{h , j} {e}^{{\hat{s}}_{j}} \\ = {\sum }_{h} \frac{{m}_{h , i} {\hat{r}}_{i} {m}_{h , j} {\hat{r}}_{j}}{{ρ}_{h}^{2}} \\ = {\sum }_{h} \frac{{m}_{h , i} {\hat{r}}_{i}}{{ρ}_{h}} \frac{{m}_{h , j} {\hat{r}}_{j}}{{ρ}_{h}}\end{matrix}$$

i=j

$$\begin{matrix}{H}_{i , i} = {\hat{r}}_{i} {l}_{i} - {\sum }_{h} {m}_{h , i} ( \frac{{e}^{{\hat{s}}_{i}}}{{\sum }_{k} {m}_{h , k} {e}^{{\hat{s}}_{k }}} - \frac{{e}^{{\hat{s}}_{i}}}{{( {\sum }_{k} {m}_{h , k} {e}^{{\hat{s}}_{k}} )}^{2}} {m}_{h , i} {e}^{{\hat{s}}_{i}} ) \\ = {\hat{r}}_{i} {l}_{i} - {\sum }_{h} {m}_{h , i} ( \frac{{\hat{r}}_{i}}{{ρ}_{h}} - {m}_{h , i} \frac{{\hat{r}}_{i}^{2}}{{ρ}_{h}^{2}} ) \\ = {\hat{r}}_{i} {l}_{i} - {\sum }_{h} \frac{{m}_{h , i} {\hat{r}}_{i}}{{ρ}_{h}} + {\sum }_{h} {( \frac{{m}_{h , i} {\hat{r}}_{i}}{{ρ}_{h}} )}^{2}\end{matrix} \qquad$$

Using the convention that J_{i,i}=Jᵢ and letting

$${G}_{i , j} = {\sum }_{h} \frac{{m}_{h , i} {\hat{r}}_{i}}{{ρ}_{h}} \frac{{m}_{h , j} {\hat{r}}_{j}}{{ρ}_{h}}$$

for both cases when i=j and i≠j then we can write:

H=J+G

From “Numerical Recipes in C”, Press W. H., Teukolsky S. A., Vettering W. T., Flannery B. P. 2nd edition, 1992, Cambridge University Press, Section 10.6, pp 420-425, we can construct a multi-dimensional Newton's method to iteratively solve the equation J=0. The increment Δ at each step (the increments apply to ŝᵢ not to \hat{r}₁) is computed as:
HΔ=−J

The conjugate gradient technique is applicable here (as H is symmetric and definite positive). This is a technique which estimates A directly without needing to form the inverse H⁻¹ explicitly. There is a description and worked example in http://en.wikipedia.org/wild/Newton's_method_in_optimization and in Section 2.7 pp 83-89 of “Numerical Recipes in C” (all references to page and section numbers in Numerical Recipes in C refer to Press et al., NUMERICAL RECIPES IN C: THE ART OF SCIENTIFIC COMPUTING, Cambridge Univ. Press, 2d Ed., 1992, which is incorporated herein by reference). More sophisticated techniques are dealt with in Section 10.6 pp 420-425 of “Numerical Recipes in C”

Such an iterative method requires a starting point. There are a number of ways we could do this (also discussed above). One would be to set all the ŝᵢ=0 which is the same as setting \hat{r}ᵢ=1.

To arrive at another consider the special case when each unit h is mapped once or more to just one genome. Then

$$\begin{matrix}\frac{\partial L}{\partial {\hat{r}}_{i}} = {l}_{i} - {\sum }_{h} \frac{{m}_{h , i}}{{m}_{h , i} {\hat{r}}_{i}} \\ = {l}_{i} - {\sum }_{h} \frac{1}{{\hat{r}}_{i }} \\ = {l}_{i} - \frac{1}{{\hat{r}}_{i}} {\sum }_{h : {m}_{h , i} > 0} 1\end{matrix}$$

Let

$${m}_{i} = {\sum }_{h : {m}_{h , i} > 0} 1$$

and then we need to solve

$${l}_{i} - \frac{{m}_{i}}{{\hat{r}}_{i}} = 0 ;$$

that is,

$${\hat{r}}_{i} = \frac{{m}_{i}}{{l}_{i}}$$

To generalize this we redefine

$${m}_{i} = {\sum }_{h} ( \frac{{m}_{h} , i}{{\sum }_{k} {m}_{h , k}} )$$

One variation is to let the \hat{r}ᵢ range over both the actual species that are known and inner nodes on the phylogenetic tree. The m_{h,i} for these pseudo-species would be an average of the m_{h,i} for its children. This may give estimates of the probability that there is an unknown species which is in the clade. Also the sum of the nₕ where all the m_{h,i} are zero may give an estimate of how many completely unknown species there are.

The “hill climbing” technique discussed previously may be employed with appropriate modifications.

Iteration may be performed by a completely automated algorithm or may be user guided. A user may define control parameters such as an acceptable slope threshold or may be presented with interim results to set parameters for further iterations.

As well as performing optimisation of the proportions of genomes based only on the actual reads for a sample optimisation may also be performed for variants of the reads. These variants may include indels and/or substitutions and/or more complex structural/large-scale variations.

Optimisation may also be performed for the reverse complement direction of the reads or variants also as sequences may be read in either direction by a sequencing machine (known as nucleotide bias).

Each variant may be ascribed a weighting representing the likelihood of the respective variant occurring. This likelihood may be based at least in part on characteristics of the apparatus employed (it is known that different sequencing machines have different propensities for generating certain read errors).

The likelihood may also be based at least in part on the chemistry of the sample. There are known to be different likelihoods of errors in relation to different base sequences.

The sample segments (such as reads) may also be translated into another form and compared to reference segments in that form. For example the sample may comprise or consist of DNA or RNA and the DNA or RNA segments may be converted to protein segments and compared to a library of protein segments. The protein segments may thus be used to assess the prevalence of proteins encoded and/or expressed in the sample by a process comprising comparing the protein segments to a library of protein segments.

“Unmapped reads” (i.e. segments or “reads” that are not present in a library of genome segments) may also be utilised to refine proportion values. By categorising unmapped reads additional information can be obtained that can assist in refining proportions. As one example, unmapped reads can be categorised as “paired end reads” when the reads were generated by a suitable sequencing procedure. The relative amount of unmapped reads can be used to estimate the proportion value for unidentified genomes, genomes of species other than the sources of the reference genomes, or genomes from organisms that are phylogenetically distant from the sources of the reference genomes.

RNA sequences are transcribed in some cells from a series of “exons” that are not continuous on the genome, but are separated by “introns”.

Which exons are actually included during splicing can vary which leads to different “splice variants”, that is one gene can lead to multiple different RNA variants which then translate into non-identical proteins.

One important problem when analyzing RNA sequences such as metazoan, e.g. mammalian, RNA sequences, where alternative splicing and splice variants are relatively common, is to estimate the relative frequency of each of these different possible splice variants. Such splice variants are similar to species in a metagenomics sample in that a read can map to one or more different splice variants just as it might map to one or more species. Thus it is possible to estimate the frequency of the splice variants in the same way that the frequency of species or proteins is estimated.

Further processing may include assembly of two or more segments into a composite segment and analysing the composite segment against the library of genome segments. Paired end reads naturally make good candidates for assembly. Exons may also be assembled in the composite segments due to their importance. Other categorised segments may also be combined based on their phylogeny and relevance to any target genomes,

A number of algorithms may be employed to determine proportion values and the order of application may be predetermined or based upon processing metrics such as the speed of the algorithm; the precision of each algorithm; the number of segments in the sample; the number of segments in the genomes; the number of genomes; the nature of the sample; the available processing resource; and the required confidence level for the proportion values.

The proportion results may be utilised to estimate the concentration of different genomes in a sample or to provide population distribution information for a number of genomes. Concentration or “abundance” values may be obtained by estimating the concentration of all genomic material in a sample (the total amount of DNA may be calculated by using an optical spectrometry procedure that calculates the change in light passing as a function of the amount of DNA present) and utilising the proportion values to determine the concentration of a particular genome in a sample. This method may be particularly suited for use in monitoring food, air and water samples. It can also be applied to determine the change in concentration over time of a particular genome in samples from a source, e.g., a human, as discussed above.

The method may also be used to provide a fingerprint of a source. Each source may have a characteristic genomic population distribution. This may be used for food tracing etc. By obtaining a characteristic genome proportion values for one or more source and comparing this with genome proportion values for a sample an assessment can be made as to whether the sample is characteristic of a source. A sample may be considered to be characteristic of a source if comparison is within a prescribed confidence level. Characteristic genome proportion values may be tracked in a series of samples over time. Proportion values may be monitored for the appearance of a fingerprint not initially present or a change in the fingerprint over time, which can indicate a change in the state of the source, e.g., spoliation of food or acquisition or development of an infection.

Historical probability information from prior samples may also be utilised to assess correlation.

The correlation between sample genomes and reference genomes obtained by a method as described above may be graphically displayed by displaying regions of correlation between reads and reference sequences. A user may modify correlation parameters to see the change in correlation results. Parameters a user may modify may include: read length; population distribution; thresholds; and read variants. Time course data can be displayed in various ways, including a series of panels on a sheet or screen, a series of displays in the nature of a slide show, or an animation in which a graphical display of a sample from a first time morphs into a graphical display from a subsequent time, optionally with similar effects to show still further subsequent times, e.g., third, fourth, fifth, sixth, seventh, eighth, ninth, tenth, etc.

The degree of correlation may be indicated by a visual attribute of each region, such as colour.

The invention also encompasses methods of obtaining expression levels of related genes in a mixed population. This may be achieved by using a processing engine to optimise estimated proportion values for the related genes based on input data comprising the prevalence of segments of the related genes in a mixed sample, the prevalence of the segments in the genomes of the population, and the prevalence of the genomes of the population in the mixed sample, thereby producing optimised proportion values for the expression levels of the related genes in the mixed population. Much as described above with respect to genomes, samples can be obtained from a source at multiple times to allow determination of the change over time of expression levels of the related genes. The invention further encompasses a method of obtaining expression levels of related genetic pathways in a mixed population, the method comprising using a processing engine to optimise estimated proportion values for the related genetic pathways based on input data comprising the prevalence of segments of genes in the related genetic pathways in a mixed sample, the prevalence of the segments in the genomes of the population, and the prevalence of the genomes of the population in the mixed sample, thereby producing optimised proportion values for the expression levels of the related genetic pathways in the mixed population. Much as described above with respect to genomes, samples can be obtained from a source at multiple times to allow determination of the change over time of expression levels of the related genetic pathways. The populations may be, e.g., microbial populations, which may include one or more of protists, bacteria, archaea, viruses, fungi, etc. The populations may also be a sample from a multicellular organism, such as a mammal, e.g., a human, such as a human infant, which contains cells of different types (blood cells, stem or progenitor cells, connective tissue cells, neurons, glia, endothelial and epithelial cells, and organ-specific cells such as splenocytes, hepatocytes, etc.) and/or microbes, such as bacteria, that live within or on the body of the multicellular organism, including microbes or bacteria present in the gut, which may be sampled, e.g., through fecal samples.

The methods discussed in the previous paragraph may involve obtaining the prevalence of the genomes of the population in the mixed sample in the form of optimised proportion values as described previously in this specification. The methods discussed in the previous paragraph may further comprise obtaining at least one set of comparative expression levels of the related genes or the related genetic pathways in at least one additional mixed population and comparing the expression levels with the at least one set of comparative expression levels, thereby facilitating analysis of effects on the expression levels in correlation with the prevalence in the mixed population of one or more members of the mixed population. The methods discussed in the previous paragraph may also further comprise identifying a member of the mixed population whose genome comprises a gene or genetic pathway which is highly expressed and is one of the related genes or the related genetic pathways. Each of the steps described above can be performed with respect to samples obtained from a source at different times, and results from the samples may be compared to determine the change in one or more values over time, including changes in relative expression levels, prevalence in the mixed population of one or more members of the mixed population, and/or which member or members of the mixed population has a genome comprising a gene or genetic pathway which is highly expressed and is one of the related genes or the related genetic pathways. A highly expressed gene may be, e.g., a gene with above-average or above-median expression, or expression in the top quartile, decile, or percentile, in the top 1000^{th}, 10000^{th}, 100000^{th}, or the highest overall expression either in terms of absolute amount or amount relative to the proportion of the corresponding genome in the sample; so too with genetic pathways.

Systems for implementing the method of the invention may consist of small stand alone systems suited to use in the field, large systems suitable for detailed analysis, distributed systems or hybrid systems consisting of a small system that can access remote processing power as required.

A small stand alone system for determining the genomic population distribution of a physical sample may include a laptop computer and a portable sequencer, such as a nanopore sensor as produced by Oxford Nanopore Technologies Ltd to obtain reads from a sample. The laptop may have a local database of reference genome segments stored on local storage media. The laptop processor may determine proportion values of the reference genomes occurring in the sample by optimising the proportion values using methods as described above. In particular the laptop may utilise algorithms most suited to the processing capabilities of the laptop, the reference genomes stored locally and other metrics such as the number of segments in the sample; the number of segments in the genomes; the number of genomes; the nature of the sample; and the required confidence level for the proportion values.

Where the proportion values cannot be determined locally to a desired confidence level, or where additional processing is desired, further processing may be transferred to a remote computer system having greater processing power and/or a larger database of reference genomes. To reduce the amount of data required to be sent only selected reads may be sent for further processing. This could be all unmapped reads or where reads have been categorised only reads of certain categories may be sent for further processing (such as exons).

Any computer system may display results in a manner enabling visual display of the results. This could simply be a bar graph showing proportions or concentrations of monitored genomes. Alternatively a more complex display technique could be employed to facilitate exploration of the results—for example a shape could be displayed representing probability levels for different proportion values with the proportion values for different genomes displayed simultaneously. By allowing a user to navigate over the surface the user may explore different proportion values that are possible within a probability range.

There is thus provided a population based method for evaluating genomic sequences that can quickly and simultaneously provide population information for all genomes and better assess the prevalence of a target genome and changes in the prevalence of a target genome over time.

The specific examples disclosed in this specification are to be construed as merely illustrative, and not limitative of the remainder of the disclosure in any way whatsoever. Without further elaboration, it is believed that one skilled in the art can, based on the description herein, utilise the present invention to its fullest extent.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. To the extent the material incorporated by reference contradicts or is inconsistent with this specification, the specification will supersede any such material. The citation of any references herein is not an admission that such references are prior art to the present invention.

Unless otherwise indicated, all numbers used in the specification, including claims, to refer to quantitative parameters are to be understood as approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. The recitation of series of numbers with differing amounts of significant digits in the specification is not to be construed as implying that numbers with fewer significant digits given have the same precision as numbers with more significant digits given.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

Unless otherwise indicated, the terms “at least” or “one or more” preceding a series of elements are to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

The publications discussed herein are provided solely for their disclosure prior to the effective filing date of the present application. Nothing herein is to be construed as an admission that the present invention is not entitled to disqualify such publication based on prior disclosure, common ownership, derivation, or any other basis. Further, the dates of publication provided may be different from the actual publication dates which may need to be independently confirmed.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of evaluating a change in prevalence of a plurality of genomes in a source over time,
comprising using a processing engine to optimise estimated proportion values for the plurality of genomes in at least a first sample obtained from the source at a first time and a second sample obtained from the source at a second time,
based on input data comprising the prevalence of segments in at least the first and second samples and the prevalence of the segments in the plurality of genomes,
thereby producing optimised proportion values for the prevalence of the plurality of the genomes in at least the first and second samples.

2. A method of evaluating a change in prevalence of one or more reference genomes in a source over time comprising the steps of:
a. obtaining at least a first set of segments of at least a first sample obtained from the source at a first time and a second set of segments of a second sample obtained from the source at a second time;
b. identifying segments in at least the first and second sets which are contained in the one or more reference genomes; and
c. maximising a probability function based on:
i. the number of occurrences of each segment identified in step b in the one or more reference genomes, and
ii. proportion values comprising (1) one or more first proportion values of the one or more reference genomes in the first sample and (2) one or more second proportion values of the one or more reference genomes in the second sample,
by optimising the one or more first proportion values and the one or more second proportion values.

3. A method performed by one or more processors, executing program instructions stored on one or more memories, causing the one or more processors to perform the method comprising the steps of claim 1.

4. The method of claim 1, wherein the first and second samples comprise biological sequence data stored in a computer-readable medium.

5. The method of claim 1, wherein the source is a human.

6. The method of claim 1, wherein the source is a human infant.

7. The method of claim 6, wherein the human infant is premature.

8. The method of claim 6, wherein the human infant has been admitted to a neonatal intensive care unit.

9. The method of claim 1, wherein at least 2 samples were obtained per day, for a period of 2 or more days, further comprising determining proportion values for the plurality of genomes for at least 4 of the samples.

10. The method of claim 6, wherein no symptoms of bacterial infection were reported for the infant prior to taking the second sample or no symptoms of bacterial infection were observed for the infant prior to taking the second sample.

11. The method of claim 6, wherein no symptoms of infectious disease were reported for the infant prior to taking the second sample or no symptoms of infectious disease were observed for the infant prior to taking the second sample.

12. The method of claim 1, wherein at least the first and second samples are fecal samples.

13. The method of claim 1, wherein the plurality of genomes comprises at least one of a Streptococcus genome, a Serratia genome, a Clostridium genome, a Staphylococcus genome, and an Escherichia coli genome.

14. The method of claim 1, wherein the plurality of genomes comprises at least one of a Group B Streptococcus genome, a Streptococcus agalactiae genome, a Serratia marcescens genome, a Clostridium difficile genome, a coagulase-positive Staphylococcus genome, a Staphylococcus aureus genome, and an Escherichia coli pathogenic strain genome.

15. The method of claim 1, further comprising determining that the prevalence of at least one genome in the sample has increased in a later sample relative to an earlier sample.

16. The method of claim 1, further comprising determining that the prevalence of at least one genome in the sample has decreased in a later sample relative to an earlier sample.

17. The method of claim 15, wherein the at least one genome whose prevalence has increased comprises at least one pathogenic bacterial genome.

18. The method of claim 1, wherein the at least one genome whose prevalence has changed comprises a Streptococcus genome, a Serratia genome, a Clostridium genome, a Staphylococcus genome, or an Escherichia coli genome.

19. The method of claim 1, wherein the at least one genome whose prevalence has changed comprises a Group B Streptococcus genome, a Streptococcus agalactiae genome, a Serratia marcescens genome, a Clostridium difficile genome, a coagulase-positive Staphylococcus genome, a Staphylococcus aureus genome, or an Escherichia coli pathogenic strain genome.

20. The method of claim 17, further comprising administering at least one antibiotic to the source of the samples.

21. The method of claim 17, further comprising administering an effective therapeutic regimen of one or more antibiotics to the source of the samples.

22. The method of claim 1, further comprising placing the source under quarantine.

23. The method of claim 1, wherein the source is an infant, and the method further comprises isolating the source from other infants.

24. The method of claim 1, wherein a probability function is employed to optimise the proportion values.

25. The method of claim 1, wherein the estimated proportion values are optimised iteratively.

26. The method of claim 24 wherein a probability function is employed to optimise the proportion values by moving the iteration in a direction in which the derivative of the probability function is most strongly increasing.

27. The method of claim 25 wherein an iteration step size is determined based on the second derivative of the probability function.

28. The method of claim 24 wherein the probability function is a Poisson distribution.

29. The method of claim 24 wherein the negative log of a global probability function is minimised.

30. The method of claim 24 wherein the probability function is a global probability function and the first partial derivatives of the global probability function are minimised using a direct non-linear technique or a multidimensional Newtons technique.

31. The method of claim 25 wherein a Hessian matrix is used to determine iteration direction and step size.

32. The method of claim 1, wherein the input data further comprises prevalence of variants of the segments in the plurality of genomes.

33. The method of claim 32 wherein the variants of the segments comprise variants with indels or substitutions relative to the segments.

34. The method of claim 32 wherein each variant is associated with a weighting representing the likelihood of the respective variant occurring.

35. The method of claim 1, further comprising, prior to using a processing engine to optimise estimated proportion values, determining the prevalence of the segments in at least the first and second samples from frequencies of the segments in sequencing data from at least the first and second samples.

36. The method of claim 1, wherein segments that do not correlate with the plurality of genomes (“unmapped segments”) are categorised as paired end reads or not paired end reads and categorisation information is utilised to refine proportion values.

37. The method of claim 1, wherein a plurality of algorithms is employed to determine proportion values.

38. The method of claim 37 wherein the algorithms are applied in an order depending upon one or more processing metrics comprising: the speed of each algorithm; the precision of each algorithm; the number of segments in the sample; the number of segments in the genomes; the number of genomes; the nature of the sample; the available processing resource; and the required confidence level for the proportion values.

39. The method of claim 1, wherein at least the first and second samples comprise DNA segments, the DNA segments are converted to protein segments, and the prevalence of proteins encoded in at least the first and second samples is determined by comparing the converted protein segments to a library of protein segments.

40. The method of claim 38 further comprising determining the abundance of one or more genomes in at least the first and second samples based on the optimised proportion values and the concentration of genomic material in at least the first and second samples.
